(19) United States
(12) Patent Application Publication
(10) Pub. No.: US 2021/0065879 A1
(43) Pub. Date: Mar. 4, 2021

(54) INFORMATION PROCESSING APPARATUS AND INFORMATION PROCESSING METHOD

(71) Applicant: Konica Minolta, Inc., Tokyo (JP)

(72) Inventor: Seiji NOMURA, Tokyo (JP)

(21) Appl. No.: 16/993,899

(22) Filed: Aug. 14, 2020

(30) Foreign Application Priority Data
Aug. 29, 2019 (JP) 2019-156244

(51) Int. Cl. G16H 30/20; G16H 30/40; G06F 16/11

(52) U.S. Cl. CPC G16H 30/20; G06F 16/116; G16H 30/40

(57) ABSTRACT

An information processing apparatus includes a hardware processor. The hardware processor extracts an examination-related string from an examination-related document file, converts the examination-related document file into image data, and attaches the extracted examination-related string to the image data, thereby creating a file in a DICOM format.

### CROSS-REFERENCE TO RELATED APPLICATIONS

The entire disclosure of Japanese Patent Application No. 2019-156244 filed on Aug. 29, 2019 is incorporated herein by reference in its entirety.

### BACKGROUND

### Technological Field

The present disclosure relates to an information processing apparatus and an information processing method.

### Description of the Related Art

In a medical setting, when a doctor refers to and makes a diagnosis from an examination image(s) that has been generated by photographing a patient, the doctor also checks documents, such as a report and a consent form, on an examination in which the examination image has been generated.

As a technology for preparation of reports on images, for example, a system for assisting preparation of interpretation reports is proposed in JP 2009-259106 A. The system collects, on the basis of strings or annotation data associated with images, key images related thereto.

As another example, a report preparing apparatus is proposed in JP 2011-141837 A. The apparatus reproduces comment information associated with an examination image(s) in a findings section of a report.

### SUMMARY

However, examination-related document files, such as reports and consent forms, are usually PDF (Portable Document Format) files, which are different in format from DICOM (Digital Image and Communications in Medicine) files of examination images. Hence, in order to check document files, it is necessary to operate a viewer for displaying document files, the viewer being different from an image viewer for displaying image files. Thus, complicated operations are mandatory. Further, because document files and examination image files cannot be checked on the same screen, presence of document files may not be noticed. Also, when document files are displayed on a screen different from a screen where examination image files are displayed, the screen where document files are displayed may interfere with browsing of examination images displayed on the different screen.

Further, in order to make a PDF file(s) handleable by a DICOM image viewer, there is used a method of converting a PDF file into a JPEG file, thereby generating image data, and attaching supplementary information to the image data. However, this method requires that examination information of an examination corresponding to the PDF file be obtained and operations of the supplementary information be made. Thus, it is difficult to automatically associate a document file(s) on an examination with the examination or with an examination image(s) generated in the examination.

The present disclosure has been made in view of the above problems of the conventional technologies, and objects of the present disclosure include associating a document file(s) on an examination with the examination.

In order to achieve at least one of the abovementioned objects, according to an aspect of the present disclosure, there is provided an information processing apparatus including a hardware processor that:

extracts an examination-related string from an examination-related document file;

converts the examination-related document file into image data; and

attaches the extracted examination-related string to the image data, thereby creating a file in a DICOM format.

In order to achieve at least one of the abovementioned objects, according to another aspect of the present disclosure, there is provided an information processing method comprising:

extracting an examination-related string from an examination-related document file;

converting the examination-related document file into image data; and

attaching the extracted examination-related string to the image data, thereby creating a file in a DICOM format.

### BRIEF DESCRIPTION OF THE DRAWINGS

The advantages and features provided by one or more embodiments of the present invention will become more fully understood from the detailed description given hereinbelow and the appended drawings that are given by way of illustration only, and thus are not intended as a definition of the limits of the present invention, wherein:

FIG. 1 shows an example of configuration of a medical information management system according to an embodiment(s) of the present disclosure;

FIG. 2 is a block diagram showing a functional configuration of an image storage apparatus;

FIG. 3 shows a folder structure where data are stored in a file storage region;

FIG. 4 is a block diagram showing a functional configuration of a document file registration apparatus;

FIG. 5 is a block diagram showing a functional configuration of a viewer terminal;

FIG. 6 is a flowchart showing a document file registration process that is performed by the document file registration apparatus;

FIG. 7 shows an example of a document file;

FIG. 8 shows an example of a text file into which the document file has been converted;

FIG. 9 shows an example of a setting file;

FIG. 10 shows examples of strings extracted from the text file on the basis of the setting file;

FIG. 11 is an image diagram of a search process that is performed by the document file registration apparatus with respect to the image storage apparatus;

FIG. 12 shows an image diagram of a DICOM document file;

FIG. 13 is a ladder chart showing an examination browsing process that is performed by the viewer terminal and the image storage apparatus; and

FIG. 14 shows an example of an examination browsing screen displayed on the viewer terminal.

### DETAILED DESCRIPTION OF EMBODIMENTS

Hereinafter, one or more embodiments of an information processing apparatus of the present invention will be described with reference to the drawings. However, the scope of the present invention is not limited to the disclosed embodiments or illustrated examples.

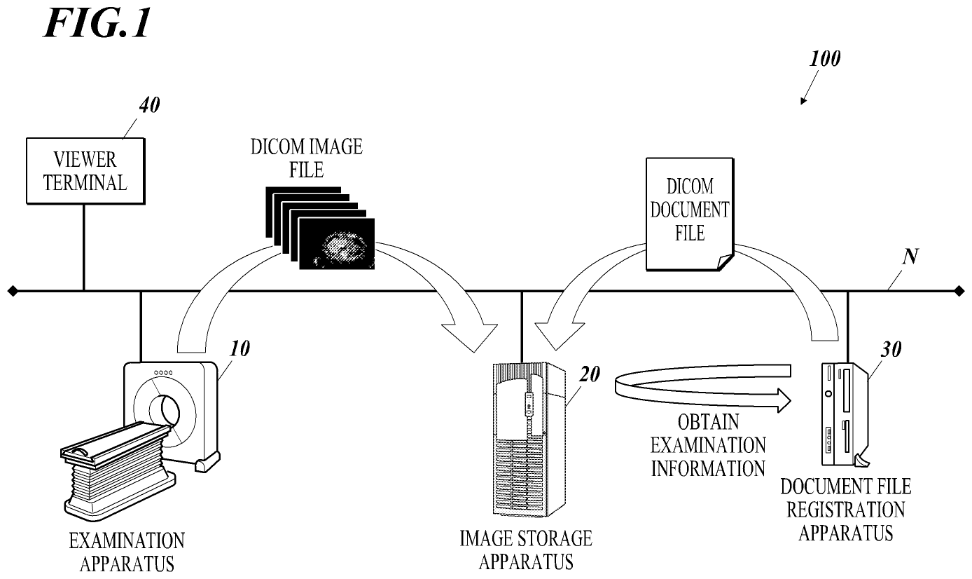

FIG. 1 shows an example of configuration of a medical information management system 100.

As shown in FIG. 1, the medical information management system 100 includes: an examination apparatus 10; an image storage apparatus 20; a document file registration apparatus 30 as an information processing apparatus; and a viewer terminal 40. These apparatuses are connected to one another so as to be able to send and receive data via a communication network N, such as a LAN (local Area Network) and/or a WAN (Wide Area Network). The apparatuses constituting the medical information management system 100 are in conformity with HL7 (Health Level Seven) standard and DICOM standard, and communicate with one another in accordance with the HL7 and DICOM standards.

The examination apparatus 10 is a modality, such as an X-ray imaging apparatus (DR, CR), an ultrasonic diagnostic apparatus (US), a CT or an MRI, and generates image data of examination images by photographing patients. In accordance with the DICOM standard, the examination apparatus 10 writes supplementary information (patient information, examination information, etc.) in the header of an image file of an examination image, thereby attaching the supplementary information to the examination image and generating a DICOM image file. DICOM image files are sent to the image storage apparatus 20, and stored in and managed by the image storage apparatus 20.

The image storage apparatus 20 manages examination-related files in the DICOM format (which hereinafter may be referred to as DICOM files) by examination. The image storage apparatus 20 stores DICOM image files generated by the examination apparatus 10 and DICOM document files created by the document file registration apparatus 30, and stores and manages these by patient and examination Examples of the image storage apparatus 20 include a PACS (Picture Archiving and Communication System).

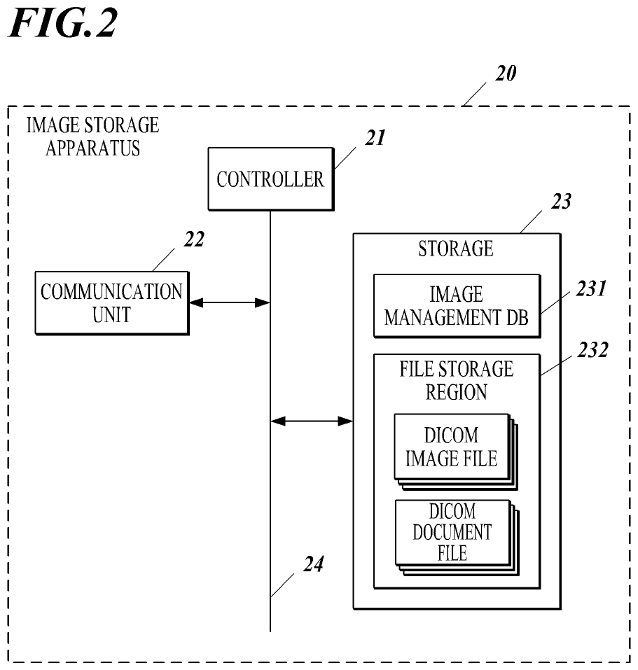

FIG. 2 shows a functional configuration of the image storage apparatus 20.

As shown in FIG. 2, the image storage apparatus 20 includes a controller 21, a communication unit 22 and a storage 23. These components are connected to one another via a bus 24.

The controller 21 includes a CPU (Central Processing Unit), a ROM (Read Only Memory) and a RAM (Random Access Memory), and comprehensively controls operation of each component of the image storage apparatus 20. More specifically, the CPU reads out various process programs stored in the ROM, loads the read programs into the RAM, and performs various processes in cooperation with the loaded programs.

The communication unit 22 is constituted by a network interface or the like, and sends and receives data to and from external apparatuses connected via the communication network N. For example, the communication unit 22 receives examination images (DICOM image files) generated by the examination apparatus 10 photographing patients. The communication unit 22 also receives DICOM document files created by the document file registration apparatus 30.

The storage 23 is constituted by an HDD (Hard Disk Drive), a nonvolatile memory and/or the like, and stores various data. The storage 23 stores an image management DB (DataBase) 231. Also, the storage 23 has a file storage region 232.

The image management DB 231 stores, for each DICOM file stored in the file storage region 232, supplementary information (header information) so as to be retrievable. The supplementary information includes patient information, examination information, series information and image information.

The patient information includes a patient ID, a patient's name, a patient's birth date and a patient's sex. The patient ID is identification information for identifying a patient.

The examination information includes an examination instance UID (study instance UID) and an examination date and time. The examination instance UID is identification information for identifying an examination and has uniqueness guaranteed by the DICOM standard.

The series information includes a series instance UID, a modality and a site (part of body). The series instance UID is identification information for identifying a series and has uniqueness guaranteed by the DICOM standard.

The image information includes an SOP instance UID, a generation date and time, an imaging direction and a file path indicating a file-stored location. The SOP instance UID is identification information for identifying a DICOM file and has uniqueness guaranteed by the DICOM standard.

The file storage region 232 stores DICOM image files received from the examination apparatus 10, DICOM document files received from the document file registration apparatus 30, and so forth.

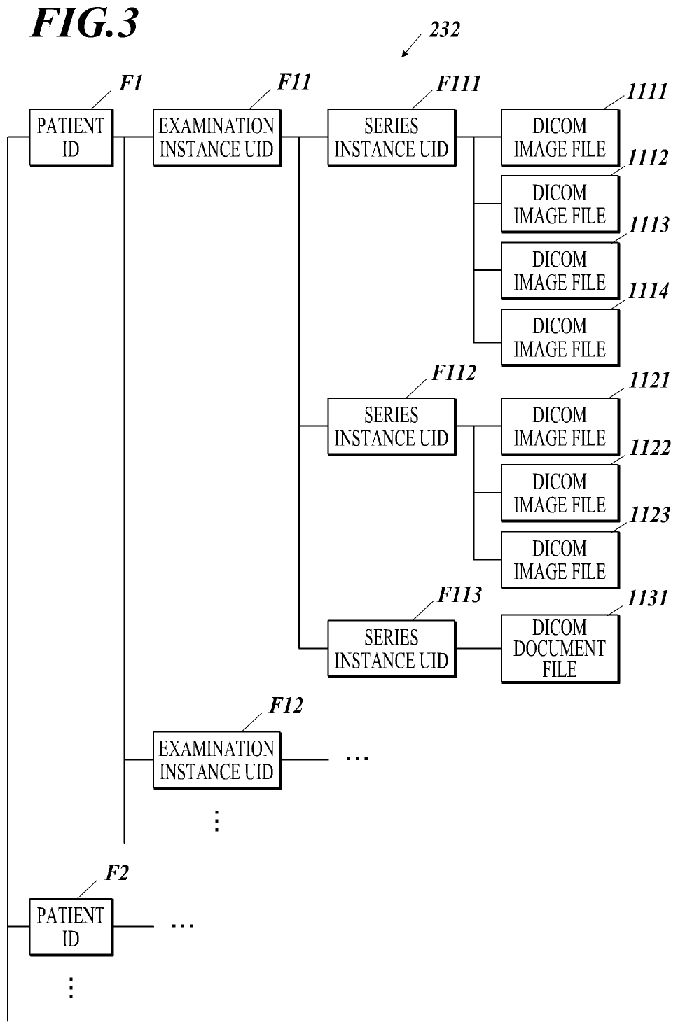

FIG. 3 shows a folder structure where data are stored in the file storage region 232 of the storage 23. Data are stored in folders hierarchized in order of patient, examination and then series.

More specifically, “Patient ID” folders F1, F2, . . . are created for respective patients.

As subfolders of the “Patient ID” folder F1, “Examination Instance UID” folders F11, F12, . . . are created for respective examinations. Similarly, as subfolders of the “Patient ID” folder for another patient, “Examination Instance UID” folders are created.

As subfolders of the “Examination Instance UID” folder F11, “Series Instance UID” folders F111, F112, F113 are created for respective series. Similarly, as subfolders of the “Examination Instance UID” folder for another examination, “Series Instance UID” folders are created.

The “Series Instance UID” folder F111 stores DICOM image files 1111 to 1114.

The “Series Instance UID” folder F112 stores DICOM image files 1121 to 1123.

The “Series Instance UID” folder F113 stores a DICOM document file 1131.

The number of files stored in each folder is not limited to the illustrated examples.

Similarly, a “Series Instances UID” folder(s) for another (other) series stores a DICOM image file(s) or a DICOM document file(s).

Uniqueness is not guaranteed for patient IDs by the DICOM standard, and hence there are some systems that do not have a layer of patients identified by patient IDs.

The controller 21 sends screen data to the viewer terminal 40 when receiving a request for an examination image(s) and/or a document(s) from the viewer terminal 40 through the communication unit 22, the screen data for displaying the requested examination image(s) and/or document(s).

The controller 21 causes a display 42 (shown in FIG. 5) of the viewer terminal 40 to display, on the same screen, DICOM files (DICOM image file(s) and DICOM document file(s)) on (related to) an examination selected on an operation unit 43 (shown in FIG. 5) of the viewer terminal 40.

The document file registration apparatus 30 converts examination-related document files into files in formats registerable in the image storage apparatus 20. The document file registration apparatus 30 is a computer apparatus, such as a PC (Personal Computer).

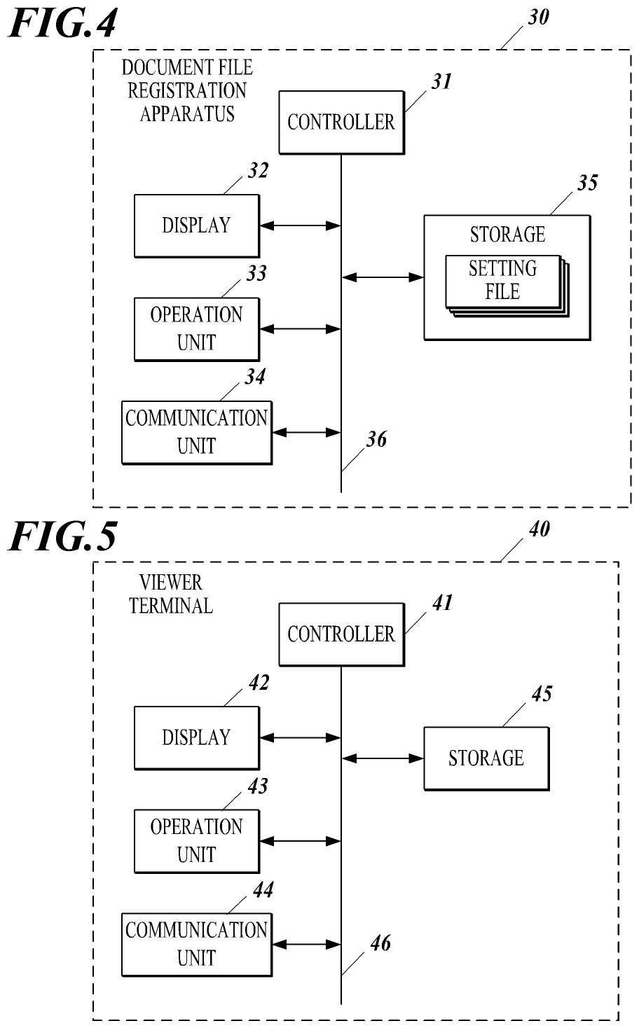

FIG. 4 shows a functional configuration of the document file registration apparatus 30.

As shown in FIG. 4, the document file registration apparatus 30 includes a controller 31 (hardware processor), a display 32, an operation unit 33, a communication unit 34 and a storage 35. These components are connected to one another via a bus 36.

The controller 31 includes a CPU, a ROM and a RAM, and comprehensively controls operation of each component of the document file registration apparatus 30. More specifically, the CPU reads out various process programs stored in the ROM, loads the read programs into the RAM, and performs various processes in cooperation with the loaded programs.

The display 32 includes a monitor, such as an LCD (Liquid Crystal Display), and displays various screens in accordance with commands of display signals input from the controller 31.

The operation unit 33 includes: a keyboard including cursor keys, character/number input keys and various function keys; and a pointing device, such as a mouse, and outputs, to the controller 31, operation signals input by a user operating the keys of the keyboard or the mouse.

The communication unit 34 is constituted by a network interface or the like, and sends and receives data to and from external apparatuses connected via the communication network N.

The storage 35 is constituted by an HDD, a nonvolatile semiconductor memory and/or the like, and stores various data. For example, the storage 35 stores setting files.

Setting files are each a file in which marker strings for extracting each examination-related string from a document file are defined. The marker strings are strings used as signs specifying extraction positions. Setting files are prepared in advance for respective types of document file (report, consent form, etc.), which is a process target.

The controller 31 extracts examination-related strings from an examination-related document file(s).

Examinations are for checking for suitability (whether specimens and/or images are suitable as an examination target), presence or absence of abnormalities/wrongness, and/or the like. Examples of the examinations include diagnostic imaging examinations.

Examination-related document files are each electronic data of a document, such as a report written on an examination image(s) generated in an examination or a consent form signed by a patient for an examination before the examination A document file may contain a diagnosis result based on an examination and/or treatment details. Document files may each be a file in the form of, other than PDF, Microsoft Word®, Microsoft Excel® or Microsoft PowerPoint®, for example. Document files may each be a file obtained by a scanner or the like reading a document printed on paper.

Examination-related strings are each a character string containing information of an examination. Characters include symbols. Examples of the examination-related strings include: patient information (patient ID, patient's name, patient's birth date, patient's age, etc.) of a patient of the examination target; examination information (examination date, etc.) of an examination; and series information (modality, etc.) of a series included in the examination.

More specifically, the controller 31 converts a document file into a text file and extracts strings (examination-related strings) from the text file.

The controller 31 identifies specific marker strings in the text file and extracts a string between the identified marker strings as an examination-related string.

The controller 31 converts the document file into image data.

The image data is data generated by converting the document file. Examples of the image data include DICOM image data.

The controller 31 attaches the extracted string(s) to the image data, thereby creating a file in the DICOM format (DICOM document file).

The controller 31 obtains, on the basis of the extracted string(s), examination identification information for identifying an examination, and attaches the obtained examination identification information to the image data. As the examination identification information, the examination instance UID is used, for example.

The controller 31 sends the created DICOM file to the image storage apparatus 20 through the communication unit 34.

The viewer terminal 40 is a computer apparatus, such as a PC, used by a doctor. The doctor browses examination images and examination-related documents on the viewer terminal 40.

FIG. 5 shows a functional configuration of the viewer terminal 40.

As shown in FIG. 5, the viewer terminal 40 includes a controller 41, a display 42, an operation unit 43, a communication unit 44 and a storage 45. These components are connected to one another via a bus 46.

The controller 41 includes a CPU, a ROM and a RAM, and comprehensively controls operation of each component of the viewer terminal 40. More specifically, the CPU reads out various process programs stored in the ROM, loads the read programs into the RAM, and performs various processes in cooperation with the loaded programs.

The display 42 includes a monitor, such as an LCD, and displays various screens in accordance with commands of display signals input from the controller 41.

The operation unit 43 includes: a keyboard including cursor keys, character/number input keys and various function keys; and a pointing device, such as a mouse, and outputs, to the controller 41, operation signals input by the user (doctor) operating the keys of the keyboard or the mouse.

The communication unit 44 is constituted by a network interface or the like, and sends and receives data to and from external apparatuses connected via the communication network N.

The storage 45 is constituted by an HDD, a nonvolatile semiconductor memory and/or the like, and stores various data.

Next, operation of the medical information management system 100 will be described.

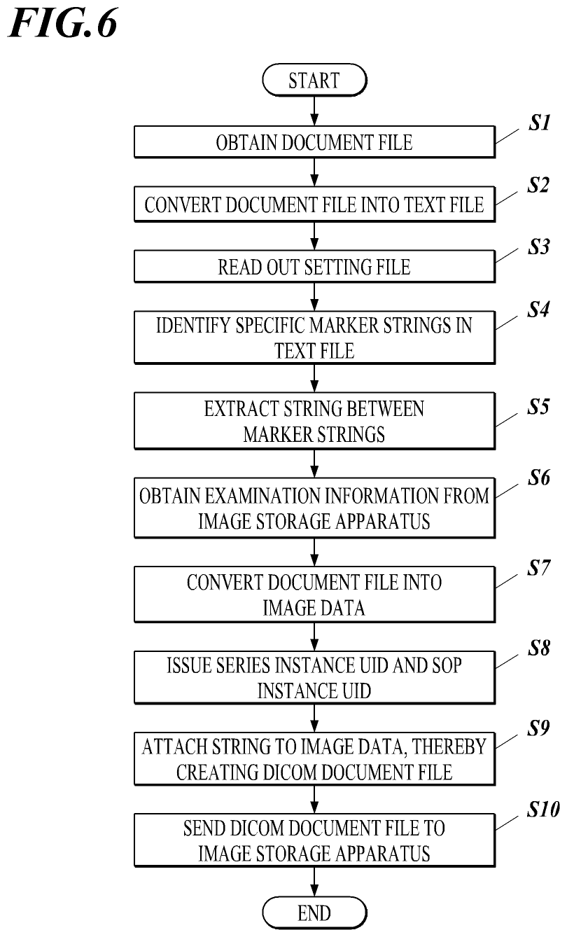

FIG. 6 is a flowchart showing a document file registration process that is performed by the document file registration apparatus 30.

First, the controller 31 obtains a document file (e.g. PDF file) (Step S1). The controller 31 may read out a document file created in the document file registration apparatus 30 from the storage 35 or may receive a document file created in an external apparatus through the communication unit 34.

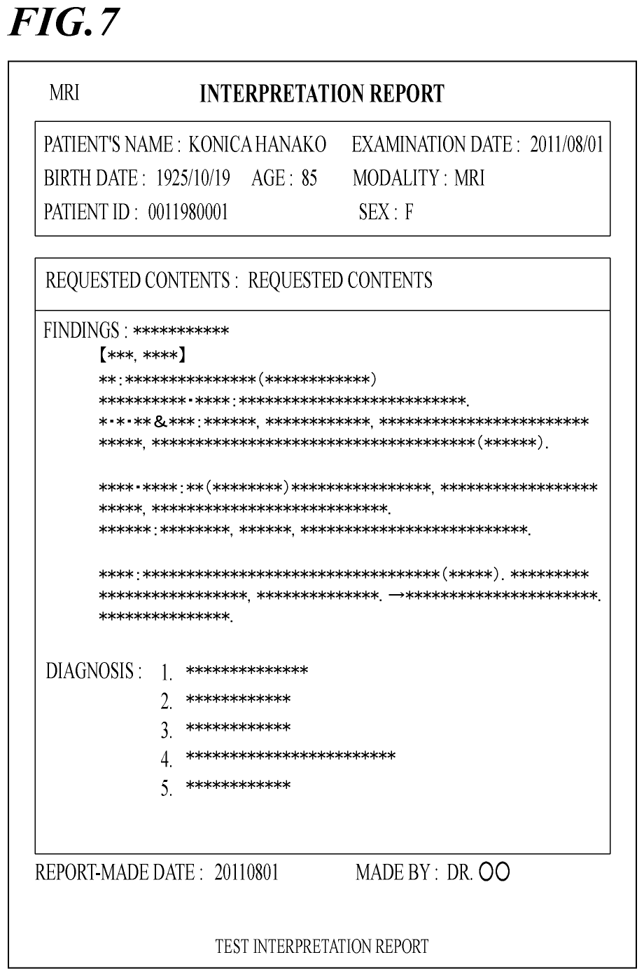

FIG. 7 shows an example of the document file (PDF file). This document file is an interpretation report on examination images taken by an MRI apparatus (examination apparatus 10).

Next, the controller 31 converts the document file into a text file (Step S2). When the document file does not contain character information, for example, when the document file is a PDF file in which character information is not embedded, the controller 31 converts the document file into a text file by using an OCR (Optical Character Recognition) function.

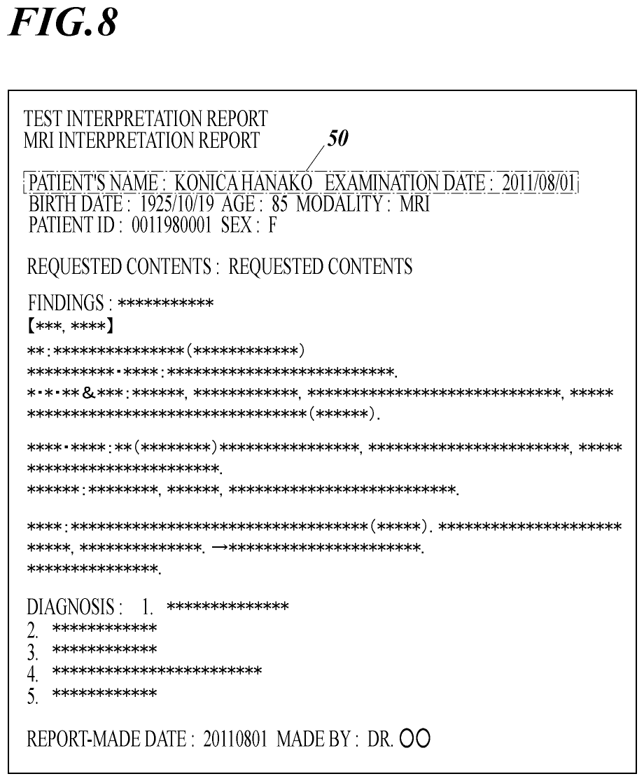

FIG. 8 shows an example of the text file into which the document file (PDF file) shown in FIG. 7 has been converted.

Next, the controller 31 reads out a setting file from the storage 35 (Step S3).

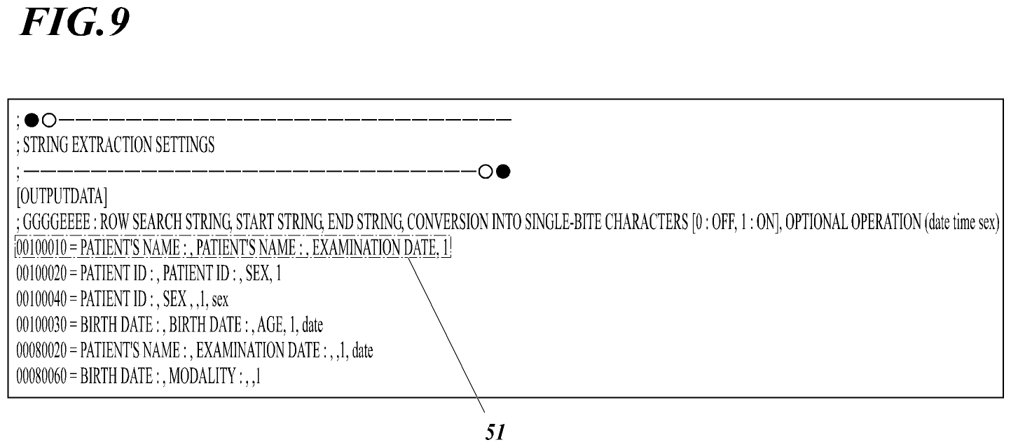

FIG. 9 shows an example of the setting file. In the setting file, extraction conditions for extracting strings from a text file (document file) are set for respective items.

In each extraction condition, the 8-digit number on the left side of an equation is a DICOM tag corresponding to an item to be extracted.

In each extraction condition, on the right side of an equation, “Row Search String”, “Start String”, “End String”, “Conversion into Single-bite Characters” and “Optional Operation” are defined in this order.

The “Row Search String” is a string for identifying, in a text file, a row from which a string (examination-related string) is extracted (hereinafter “extraction target row”). A row starting with the string written in the “Row Search String” is the extraction target row.

The “Start String” is a string (marker string) for identifying the start position of the examination-related string in the extraction target row of the text file. A string starting with a character following the start string is extracted as the examination-related string.

The “End String” is a string (marker string) for identifying the end position of the examination-related string in the extraction target row of the text file. A string ending with a character in front of the end string is extracted as the examination-related string. When characters to the last character in the extraction target row are to be extracted, the “End String” is left blank.

The “Conversion into Single-bite Characters” is information indicating whether the extracted string needs to be converted into single-bite characters. When the “Conversion into Single-bite Characters” is “1”, the extracted string needs to be converted into single-bite characters, whereas when the “Conversion into Single-bite Characters” is “0”, the extracted string does not need to be converted into single-bite characters.

The “Optional Operation” is information specifying a format when format adjustment is performed on the extracted string. For example, when a date is extracted in a specified format, format adjustment of deleting “Year”, “Month”, “Day” and/or “/”, thereby showing a date with numbers only, is performed. As another example, when a sex is extracted in a specified format, format adjustment of representing a sex with “M” or “F” is performed. When no format adjustment is to be performed, the “Optional Operation” is left blank.

Next, the controller 31 identifies specific marker strings in the text file, referring to the setting file (Step S4). More specifically, for each extraction condition (item) in the setting file, the controller 31 identifies the extraction target row from the text file in accordance with the “Row Search String”, and identifies the “Start String” and the “End String” in the extraction target row.

Next, the controller 31 extracts a string (examination-related string) between the identified marker strings (Step S5).

For example, an extraction condition 51 in the setting file shown in FIG. 9 is a condition for extracting an item (patient's name) corresponding to a DICOM tag of “(0010,0010)”.

Because the “Row Search String” in the extraction condition 51 is “Patient's Name:”, a row 50 in the text file shown in FIG. 8 is the extraction target row.

Because the “Start String” and the “End String” in the extraction condition 51 are “Patient's Name:” and “Examination Date”, respectively, “Konica Hanako”, which is a portion (string) between the “Patient's Name:” and the “Examination Date”, is extracted from the extraction target row 50.

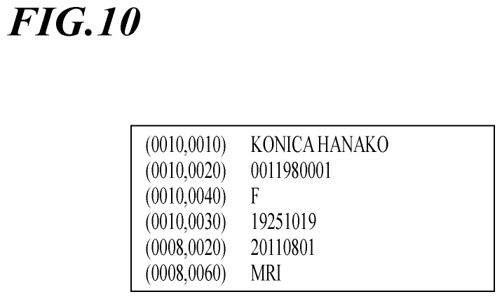

FIG. 10 shows examples of the strings extracted from the text file shown in FIG. 8 on the basis of the setting file shown in FIG. 9. In this case, a patient's name, a patient ID, a patient's sex, a patient's birth date, an examination date and a modality are extracted.

Next, the controller 31 obtains examination information (including an examination instance UID) from the image storage apparatus 20 on the basis of the extracted strings (Step S6).

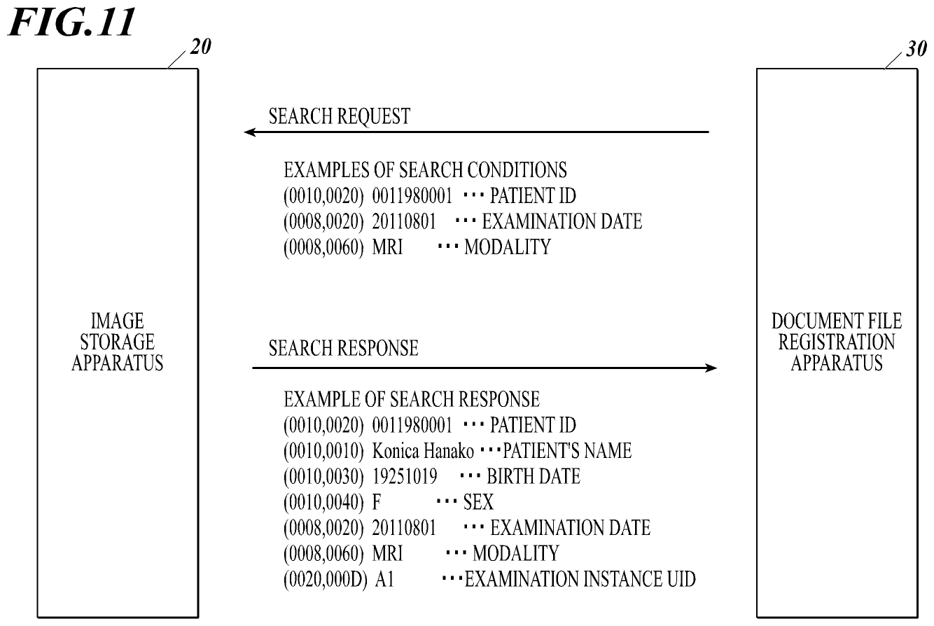

FIG. 11 is an image diagram of a search process that is performed by the document file registration apparatus 30 with respect to the image storage apparatus 20. The controller 31 of the document file registration apparatus 30 sends a search request to the image storage apparatus 20 through the communication unit 34, with, for example, a patient ID, an examination date and a modality as search conditions. The controller 21 of the image storage apparatus 20 searches the image management DB 231 and retrieves therefrom information matching the search conditions, and sends a search response (search result) to the document file registration apparatus 30 through the communication unit 22. The document file registration apparatus 30 obtains the patient ID, a patient's name, a patient's birth date, a patient's sex, the examination date, the modality and an examination instance UID from the image storage apparatus 20.

Next, the controller 31 converts the document file into image data (Step S7). The controller 31 converts the PDF file into JPEG data, for example.

Next, the controller 31 issues a series instance UID and an SOP instance UID for the image data, into which the document file as the process target has been converted, in accordance with the DICOM standard (Step S8).

Next, the controller 31 writes the strings extracted in Step S5, the strings (examination information, etc.) obtained in Step S6, and the series instance UID and the SOP instance UID issued in Step S8 into a header, thereby attaching the strings to the image data generated in Step S7 and consequently creating a file in DICOM format (DICOM document file) (Step S9).

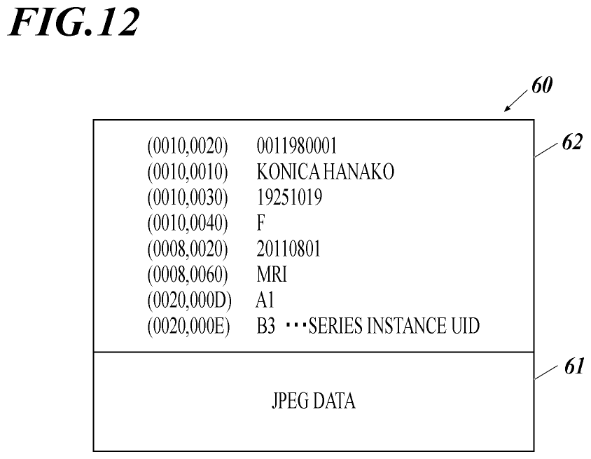

FIG. 12 is an image diagram of a DICOM document file 60 created. The DICOM document file 60 includes image data 61, such as JPEG data, and a DICOM header 62. The DICOM header 62 contains a patient ID, a patient's name, a patient's birth date, a patient's sex, an examination date, a modality, an examination instance UID and a series instance UID.

Next, the controller 31 sends the DICOM document file to the image storage apparatus 20 through the communication unit 34 (Step S10).

This completes the document file registration process.

In the image storage apparatus 20, when receiving the DICOM document file from the document file registration apparatus 30 through the communication unit 22, the controller 21 obtains the examination instance UID from the header of the DICOM document file, thereby identifying the targeted examination. The controller 21 creates, in the file storage region 232 of the storage 23, a “Series Instance UID” folder having the name of the series instance UID contained in the header of the DICOM document file as a subfolder of the “Examination Instance UID” folder for the specified examination, and stores the DICOM document file in/under this “Series Instance UID” folder. The controller 21 also obtains various types of information from the header of the received DICOM document file, and registers the obtained information in the image management DB 231.

When two or more document files on one examination are present, the document files may be managed as separate series, or the document files, which are on the same examination, may be managed as a single series. In the latter case, when receiving a search request from the document file registration apparatus 30, the image storage apparatus 20 determines whether a series for managing document files on the requested examination is present, and if the image storage apparatus 20 determines that a series for managing document files on the requested examination is already present, the image storage apparatus 20 sends the series instance UID for the series to the document file registration apparatus 30.

An issue rule(s) of the “Series Instance UID” may be set down for each type of document file, so that document files of different types can be stored and managed as different/separate series.

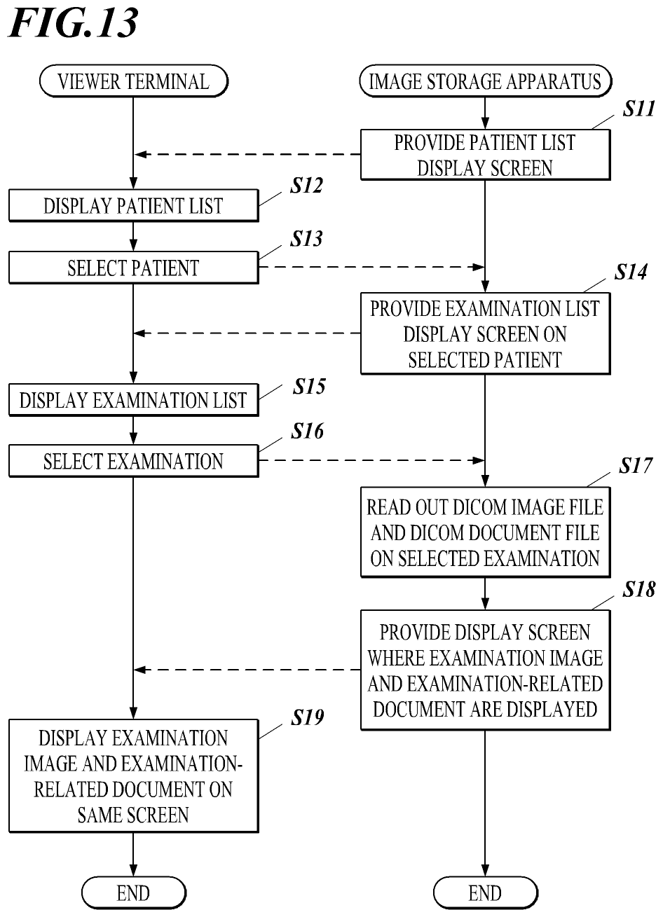

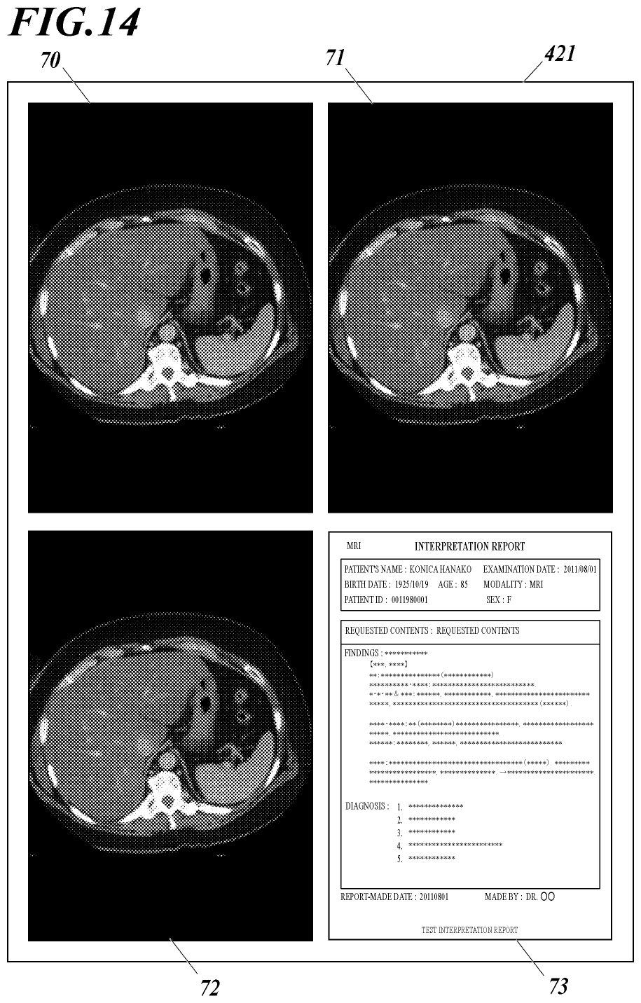

FIG. 13 is a ladder chart showing an examination browsing process that is performed by the viewer terminal 40 and the image storage apparatus 20.

First, in the image storage apparatus 20, the controller 21 refers to the image management DB 231 in the storage 23, generates a patient list display screen on the basis of the registered patient information of patients, and provides the display screen to the viewer terminal 40 through the communication unit 22 (Step S11).

In the viewer terminal 40, the display 42 displays a patient list (Step S12).

When a user of the viewer terminal 40 selects a patient from the patient list by operating the operation unit 43 of the viewer terminal 40 (Step S13), in the image storage apparatus 20, the controller 21 refers to the image management DB 231 in the storage 23, generates an examination list display screen on the basis of the examination information on the selected patient, and provides the display screen to the viewer terminal 40 through the communication unit 22 (Step S14).

In the viewer terminal 40, the display 42 displays an examination list (Step S15).

When the user of the viewer terminal 40 selects an examination from the examination list by operating the operation unit 43 of the viewer terminal 40 (Step S16), in the image storage apparatus 20, the controller 21 reads out a DICOM image file(s) and a DICOM document file(s) on the selected examination (Step S17). More specifically, the controller 21 reads out, from the file storage region 232 of the storage 23, a DICOM image file(s) and a DICOM document file(s) stored in/under (in a subfolder(s) of) the “Examination Instance UID” folder for the selected examination.

The controller 21 generates a display screen where an examination image(s) based on the DICOM image file(s) and an examination-related document(s) based on the DICOM document file(s) are displayed, and provides the display screen to the viewer terminal 40 through the communication unit 22 (Step S18).

In the viewer terminal 40, the display 42 displays the examination image(s) and the examination-related document(s) on the same screen (Step S19).

FIG. 14 shows an example of an examination browsing screen 421 displayed on the display 42 of the viewer terminal 40. The examination browsing screen 421 contains examination images 70, 71, 72 and an examination-related document 73 on a selected examination.

This completes the examination browsing process.

As described above, according to this embodiment, the document file registration apparatus 30 extracts an examination-related string(s) from an examination-related document file, converts the examination-related document file into image data, and attaches the extracted examination-related string to the image data, thereby creating a file in a DICOM format. This can associate a document file(s) on an examination with the examination.

More specifically, a document file is once converted into a text file, so that an examination-related string(s) can be easily extracted from the text file. Further, specific marker strings (strings indicating positions in front of and behind each string to be extracted) are set in advance, so that values of desired items can be obtained for sure. Further, setting files are prepared for respective types of document file, so that DICOM files suitable for types of document file can be created, not depending on the type/function of the document file registration apparatus 30.

Further, the document file registration apparatus 30 obtains examination identification information (examination instance UID) from the image storage apparatus 20 on the basis of the extracted examination-related string(s), and attaches the obtained examination identification information to the image data, into which the examination-related document file has been converted. This can easily associate a document file(s) on an examination with the examination.

Further, the document file registration apparatus 30 converts the examination-related document file into a file in a format (DICOM document file) that can be handled by the image storage apparatus 20, and then sends the file to the image storage apparatus 20. Consequently, the image storage apparatus 20 can manage DICOM document files by examination, as with examination images (DICOM image files). Thus, the image storage apparatus 20 can store a DICOM document file(s) on an examination by being associated with an examination image(s) on the same examination. The image storage apparatus 20 causes the viewer terminal 40 to display a DICOM image file(s) and a DICOM document file(s) on a selected examination on the same screen. This can prevent DICOM document files (data that have been document files) from being overlooked (not being noticed).

The above embodiment is merely an example of the information processing apparatus of the present disclosure, and hence not intended to limit the present invention. The detailed configuration and detailed operation of each component of the information processing apparatus can also be appropriately modified without departing from the scope of the present invention.

For example, in the document file registration process (shown in FIG. 6), if an examination instance UID is extractable from a document file, Step S6 may be omitted.

Further, depending on the imaging state of the examination apparatus 10 taking an examination image(s), the document file registration apparatus 30 may start the document file registration process before the image storage apparatus 20 captures (takes in) an examination image(s) (DICOM image file(s)). In this case, the document file registration apparatus 30 may monitor the image storage apparatus 20 to find out whether or not the image storage apparatus 20 has captured a DICOM image file(s) on an examination, and perform Step 6 and the subsequent steps of the document file registration process after the image storage apparatus 20 captures the DICOM image file(s).

Further, values (disease name, etc.) of predetermined items extracted from a document file may be used to collect a DICOM document file(s) and a DICOM image file(s) related thereto from the image storage apparatus 20.

Further, in the above, a ROM is used as a computer readable medium storing the programs for performing the processes. However, this is not a limit. As the computer readable medium, a nonvolatile memory, such as a flash memory, or a portable storage medium, such as a CD-ROM, can also be used. Further, as a medium to provide data of the programs via a communication line, a carrier wave can be used.

Although one or more embodiments of the present invention have been described and illustrated in detail, the disclosed embodiments are made for purposes of not limitation but illustration and example only. The scope of the present invention should be interpreted by terms of the appended claims

What is claimed is:

1. An information processing apparatus comprising a hardware processor that:
extracts an examination-related string from an examination-related document file;
converts the examination-related document file into image data; and
attaches the extracted examination-related string to the image data, thereby creating a file in a DICOM format.

2. The information processing apparatus according to claim 1, wherein the hardware processor converts the examination-related document file into a text file, and extracts the examination-related string from the text file.

3. The information processing apparatus according to claim 2, wherein the hardware processor identifies specific marker strings in the text file, and extracts the examination-related string between the identified marker strings.

4. The information processing apparatus according to claim 1, wherein the hardware processor obtains examination identification information for identifying an examination based on the extracted examination-related string, and attaches the obtained examination identification information to the image data.

5. The information processing apparatus according to claim 1,
wherein the information processing apparatus is data-communicably connected to an image storage apparatus that manages files in the DICOM format by examination, and
wherein the information processing apparatus further comprises a communication unit that, together with the hardware processor, sends the created file in the DICOM format to the image storage apparatus.

6. An information processing method comprising:
extracting an examination-related string from an examination-related document file;
converting the examination-related document file into image data; and
attaching the extracted examination-related string to the image data, thereby creating a file in a DICOM format.
